(19) United States
(12) Patent Application Publication
(10) Pub. No.: US 2024/0379232 A1
(43) Pub. Date: Nov. 14, 2024

(54) PREDICTIVE MODELING OF THERAPEUTIC AGENT RESPONSE USING DEEP LEARNING ANALYSIS OF PRE-TREATMENT AND INTRA-TREATMENT SERIAL IMAGING

(71) Applicant: ONC.AI, INC., San Carlos, CA (US)

(72) Inventors: Petr Jordan, Emerald Hills, CA (US); Rita Ciaravino, Grosse Pointe Farms, MI (US)

(21) Appl. No.: 18/657,383

(22) Filed: May 7, 2024

Related U.S. Application Data
(60) Provisional application No. 63/465,460, filed on May 10, 2023.

(51) Int. Cl. G16H 50/20

(52) U.S. Cl. CPC G16H 50/20

(57) ABSTRACT

A system and method of using pre-treatment and intra-treatment serial imaging in at least one of predictive modeling or multi-modal predictive modeling of therapeutic agent response. The method includes acquiring pre-treatment features of one or more target lesions associated with a pre-treatment scan of a target subject prior to treating the target subject according to a treatment plan. The method includes determining a set of features indicative of a change in the one or more target lesions using the pre-treatment features. The method includes providing the set of features to one or more predictive models trained to predict therapeutic agent responses based on features of target lesions. The method includes generating a predicted treatment response score to for the treatment plan based on the set of features and the one or more predictive models prior to treating the target subject according to the treatment plan.

### CROSS-REFERENCE TO RELATED APPLICATIONS

This application claims the benefit of U.S. Provisional Application No. 63/465,460 entitled “PREDICTIVE MODELING OF THERAPEUTIC AGENT RESPONSE USING DEEP LEARNING ANALYSIS OF PRE-TREATMENT AND INTRA-TREATMENT SERIAL IMAGING,” filed May 10, 2023, the disclosure of which is incorporated herein by reference in its entirety.

### TECHNICAL FIELD

The present disclosure relates to predicting therapeutic agent response using deep learning analysis, and in particular to systems and methods for using pre-treatment and intra-treatment serial imaging in at least one of predictive modeling of therapeutic agent response or multi-modal predictive modeling of therapeutic agent response.

### BRIEF DESCRIPTION OF THE DRAWINGS

The present disclosure will be understood more fully from the detailed description given below and from the accompanying drawings of various implementations of the disclosure.

FIG. 1 is a diagram showing a machine learning system for use with embodiments of the present disclosure;

FIG. 2 is a diagram showing the patient imaging collection and treatment timeline, according to some embodiments;

FIG. 3 depicts a flow diagram of a method of predicting therapeutic agent response using deep learning analysis of pre-treatment and intra-treatment serial imaging, according to some embodiments; and

FIG. 4 illustrates examples of different systems that may be used to predict immunotherapy treatment using deep learning analysis, according to some embodiments.

### DETAILED DESCRIPTION

Embodiments of the present disclosure relate to the field of predicting therapeutic agent response using deep learning analysis, and in particular to systems and methods for using pre-treatment and intra-treatment serial imaging in at least one of predictive modeling or multi-modal predictive modeling of therapeutic agent response.

Predictive modeling of therapeutic agent response can be done in multiple ways. In one approach, a computing system may use at least one of one or more pre-treatment images, a set of electronic medical record (EMR) features, or lab values/measurements (e.g., from a blood sample, a urine sample, a tissue biopsy, etc.) to predict the most likely outcome of treatment with the aim of providing the physician with the ability to choose the most appropriate therapeutic option for a given patient. In another embodiment, a predictive model may be built (e.g., trained) from a set of serial (e.g., longitudinal) features acquired prior and during therapy. A serial model may be used to at least one of select optimal therapy, to make adjustments to therapy during the course of treatment, or to provide early insights and assessment of the therapeutic response. Examples of serial modeling features span many different data domains, e.g., levels of a given serum protein measured at different times, scans (e.g., computerized tomography (CT) scans) taken prior and during therapy, a patient's cognitive performance status that is evaluated at each visit, etc. In some embodiments, a scan may include radiological images (e.g., CT scan, Magnetic Resonance Imaging (MRI), etc.), but exclude slides.

In another approach, a serial CT imaging may be used to predict the overall survival (OS) of advanced stage melanoma patients treated with immunotherapy. A radiomic model may be built from CT scans acquired at two time points (e.g., baseline and during treatment) that incorporates imaging features that capture the change in tumor appearance and volume between the two points. Models that rely on the change of appearance of the tumor at two different time points tend to have higher predictive power than a model that might only incorporate tumor appearance at baseline. This observation is the key concept behind the field of delta radiomics, where delta represent the notion of imaging feature change between two imaging time points.

However, these conventional approaches for predicting therapeutic agent response rely on at least some serial imaging that is acquired during treatment; thereby making it more likely that the conventional approaches will select and start the patient on a non-optimal treatment agent. Consequently, it could then take several months for physicians to discover that the non-optimal treatment agent is failing to treat the patient's condition, which could allow the disease to progress. Furthermore, selecting the wrong treatment agent could subject the patient to adverse side effects caused by the non-optimal treatment, which the patient would not have experienced if the optimal treatment agent were initially selected. Thus, there is a long-felt need in providing a mechanism for optimizing the prediction of therapeutic agent response prior to beginning treatment.

Aspects of the present disclosure address the above-noted and other deficiencies by using pre-treatment and/or intra-treatment serial imaging for at least one of predictive modeling of therapeutic agent response or multi-modal predictive modeling of therapeutic agent response. As described in greater detail below, the embodiments of the present disclosure use serial imaging and changes in features extracted from the serial imaging data in a pre-treatment setting. There are many different scenarios in which multiple imaging time points might be available.

In one scenario, for example, a typical lung cancer workflow might begin with a diagnostic CT or positron emission tomography CT (PET-CT) image upon which the initial diagnosis is made. In the case of late stage (e.g., stage III or IV) lung cancer at diagnosis, which comprises about 40% of cases, systemic therapy (e.g., chemotherapy, immunotherapy, or targeted therapy) is indicated. Contrast enhanced CT is the clinical standard for staging and follow-up imaging. For this reason, patients may undergo diagnostic PET-CT and contrast enhanced CT scans prior to start of therapeutic plan. Thus, having multiple scans prior to therapy start presents an opportunity for the embodiments of the present disclosure to use these pre-treatment scans in order to incorporate the difference in lesion appearance (e.g., volume, diameter, etc.) into predictive models.

In another scenario, for example, a patient might be diagnosed with an early-stage disease (e.g., stage I or II) and is treated with local therapy (e.g., surgery, radiation therapy, or ablative therapy). After some period of time, follow-up (e.g., surveillance) imaging might have detected progression of disease into stage III or IV. In such scenario, there are again multiple imaging time points containing information about the rate of disease progression, which the embodiments of the present disclosure may use to improve the accuracy in predicting therapeutic agent response.

In yet another scenario, for example, a given therapeutic agent may be indicated as a second line (2L) or third line (3L) option. In October 2017, pembrolizumab (Keytruda®) became the first immunotherapy drug approved for the first-line (1L) treatment of patients with metastatic NSCLC whose tumors express a protein called PD-L1. Prior to this approval, pembrolizumab was indicated in 2L and 3L setting, meaning that patients typically received chemotherapy in the first line setting and then may have received pembrolizumab (or another PD-1 immune checkpoint inhibitor such as nivolumab or atezolumab) upon progression. In this setting, imaging data from 1L setting may serve as pre-treatment serial imaging in embodiments of the present disclosure, where the imaging data can inform response prediction to 2L therapeutic agent.

In one embodiment, the terms “target,” “target lesion,” “target subject,” etc. may refer to a nodule, lesion, tumor, metastatic mass or an anatomical structure near (within some defined proximity to) a treatment area. In another embodiment, a target may be a bony structure or bone metastasis. In yet another embodiment a target may refer to soft tissue of a patient. A target may be any defined structure or area capable of being identified and tracked (including the entirety of the patient themselves) as described herein.

Furthermore, although a therapeutic agent (e.g., programmed cell death protein 1 (PD-1) agent, Cytotoxic T lymphocyte antigen 4 (CTLA-4) agent, etc.) is frequently referred to for convenience and brevity, the embodiments disclosed herein are similarly suitable for any other method of treatment, including but not limited to other forms of immunotherapy, chemotherapy, and radiation therapy.

### 1. Machine Learning System for Predictive Modeling Therapeutic Agent Response

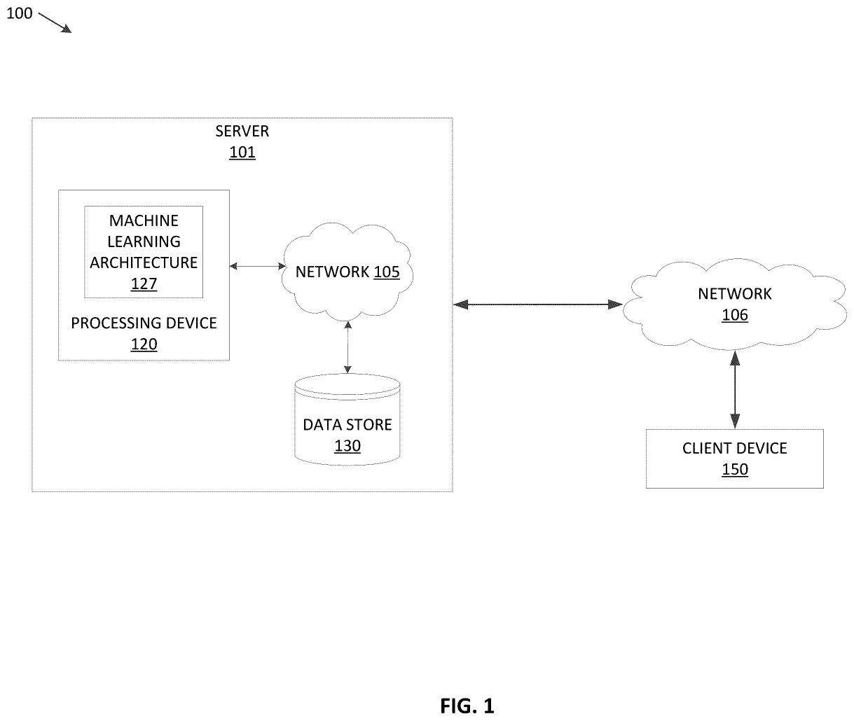

FIG. 1 is a diagram showing a machine learning system 100 for use with embodiments of the present disclosure. Although specific components are disclosed in machine learning system 100, it should be appreciated that such components are examples. That is, embodiments of the present disclosure are well suited to having various other components or variations of the components recited in machine learning system 100. It is appreciated that the components in machine learning system 100 may operate with other components than those presented, and that not all of the components of machine learning system 100 may be required to achieve the goals of machine learning system 100.

In one embodiment, system 100 includes server 101, network 106, and client device 150. Server 101 may include various components, which may allow for using at least one of pre-treatment or intra-treatment serial imaging (e.g., available on server 101, client device 150, and/or data store 130) in at least one of predictive modeling of therapeutic agent response or multi-modal predictive modeling of therapeutic agent response. Each component may perform different functions, operations, actions, processes, methods, etc., for a web application and/or may provide different services, functionalities, and/or resources for the web application. Server 101 may include machine learning architecture 127 of processing device 120 to perform operations related to using trained models to predict responses to one or more therapeutic agents using deep learning analysis of at least one of pre-treatment or intra-treatment serial imaging. In one embodiment, processing device 120 one or more graphics processing units of one or more servers (e.g., including server 101). Additional details of machine learning architecture 127 are provided with respect to the remaining figures of the present disclosure. Server 101 may further include network 105 and data store 130.

The processing device 120 and the data store 130 are operatively coupled to each other (e.g., may be operatively coupled, communicatively coupled, may communicate data/messages with each other) via network 105. Network 105 may be a public network (e.g., the internet), a private network (e.g., a local area network (LAN) or wide area network (WAN)), or a combination thereof. In one embodiment, network 105 may include a wired or a wireless infrastructure, which may be provided by one or more wireless communications systems, such as a Wi-Fi hotspot connected with the network 105 and/or a wireless carrier system that can be implemented using various data processing equipment, communication towers (e.g., cell towers), etc. The network 105 may carry communications (e.g., data, message, packets, frames, etc.) between the various components of server 101. The data store 130 may be a persistent storage that can store data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices.

Each component may include hardware such as processing devices (e.g., processors, central processing units (CPUs), graphics processing units (GPUs), memory (e.g., random access memory (RAM), storage devices (e.g., hard-disk drive (HDD), solid-state drive (SSD), etc.), and other hardware devices (e.g., sound card, video card, etc.). The server 101 may comprise any suitable type of computing device or machine that has a programmable processor including, for example, server computers, desktop computers, laptop computers, tablet computers, smartphones, set-top boxes, etc. In some examples, the server 101 may comprise a single machine or may include multiple interconnected machines (e.g., multiple servers configured in a cluster). The server 101 may be implemented by a common entity/organization or may be implemented by different entities/organizations. For example, a server 101 may be operated by a first company/corporation and a second server (not pictured) may be operated by a second company/corporation. Each server may execute or include an operating system (OS), as discussed in more detail below. The OS of a server may manage at least one of the execution of other components (e.g., software, applications, etc.) or may manage access to the hardware (e.g., processors, memory, storage devices etc.) of the computing device.

As discussed herein, the server 101 may provide machine learning functionality to a client device (e.g., client device 150). In one embodiment, server 101 is operably connected to client device 150 via a network 106. Network 106 may be a public network (e.g., the internet), a private network (e.g., a local area network (LAN) or wide area network (WAN)), or a combination thereof. In one embodiment, network 106 may include a wired or a wireless infrastructure, which may be provided by one or more wireless communications systems, such as a Wi-Fi hotspot connected with the network 106 and/or a wireless carrier system that can be implemented using various data processing equipment, communication towers (e.g., cell towers), etc. The network 106 may carry communications (e.g., data, message, packets, frames, etc.) between the various components of server 101. Further implementation details of the operations performed by server 101 are described with respect to the remaining figures of the present disclosure.

### 1.1 Machine Learning Features

Serial imaging in predictive modeling may be based on the observation that serial imaging captures changes in the appearance of lesions between pre-treatment and follow-up image, resulting from the therapeutic effect (or lack of effect) of the antineoplastic agent being administered. The embodiments of the present disclosure are centered around the observation that serial imaging performed prior to start of therapy can contain important insights about the aggressiveness (e.g., growth rate, volume, diameter) of each lesion. This is especially important in advanced stage disease with multiple tumor sites, where for example some tumor may be more stagnant, while other might exhibit aggressive growth rate. The tumor growth rate quantified from pre-treatment imaging is a powerful predictive feature that can be used in predictive models for antineoplastic agents (e.g., immunotherapy or targeted drug).

### 1.2 Response Assessment (Labels)

Once a therapeutic agent is started, some lesions might decrease in size, while some highly aggressive lesions might only decelerate in terms of growth rate. The latter (e.g., change in growth rate) may be described as the second derivative of tumor volume with respect to time and it has the potential to quantify drug effects better than the traditional change in absolute lesion diameter (e.g., the response evaluation criteria in solid tumors (RECIST) protocol. This concept can also be described as lesion kinetics, where one is concerned with measuring the acceleration vs. velocity of tumor growth. This concept can be applied to single lesion at a time or to measure an aggregate of all lesions within one patient. Furthermore, different endpoints (e.g., outcomes) can be modeled (e.g., predicted) with this approach, including those typically employed in cancer drug trials, such as the overall survival (OS), progression-free survival (PFS), overall response rate (ORR) or individual tumor kinetics (e.g., velocity, acceleration). The resulting models incorporating these novel features and assessment labels can be formulated as either classification or regression models depending on the nature of the prediction. The architecture of such models can range from simple rule-based models, decision trees, random forest, support vector machines, all the way to deep neural networks.

In one embodiment, a predictive model uses changes in features (sometimes referred to as, novel features) extracted from pre-treatment images of one or more target lesions and is trained to predict a response assessment label including, for example, RECIST or tumor volume change from baseline.

In another embodiment, the same model uses pre-treatment multi-modal features (e.g., change in blood lab values, change in urine lab values, and change in imaging features) extracted from the pre-treatment images.

In additional embodiments, the imaging and multi-modal models are trained to predict response to therapy quantified in terms of change in growth rate, which the inventors have discovered as a novel response assessment method.

### 2. Patient Imaging Collection and Treatment

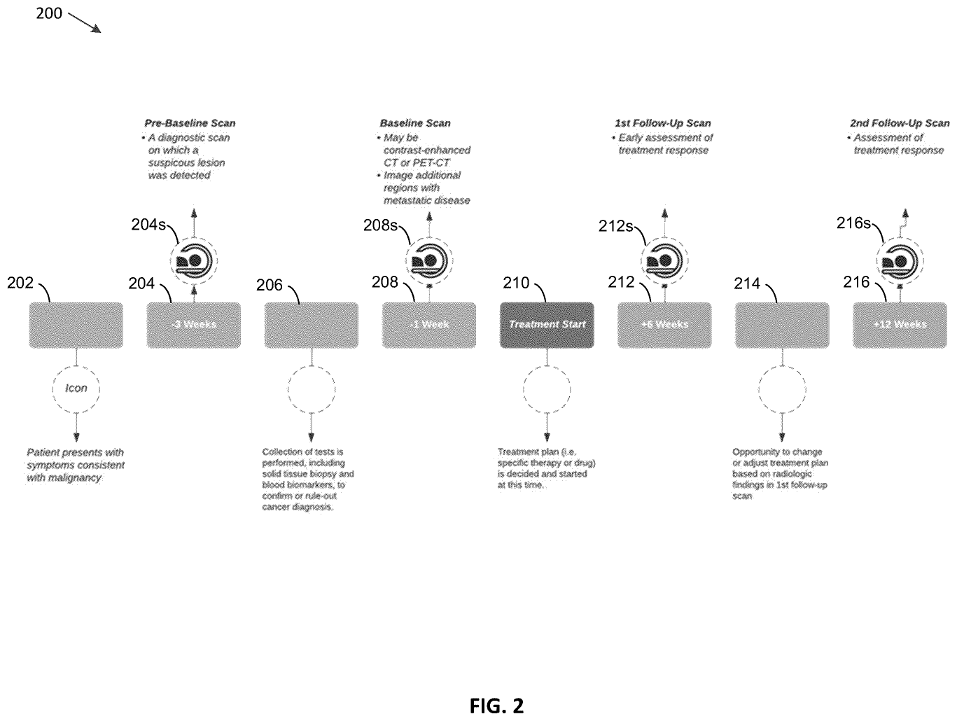

FIG. 2 is a diagram showing the patient imaging collection and treatment timeline, according to some embodiments. The diagram includes several time points (e.g., 202, 204, 206, 208) that occur pre-treatment, a time point 210 indicating when treatment starts, and several time point (e.g., 212, 214, and 216) that occur post-treatment. Although each time point is shown in FIG. 2 as being spaced apart in time by a particular time unit (e.g., −3 weeks, −1 week, etc.), the time points may be spaced apart in any time units (e.g., +/−minutes, +/−days, +/−months, etc.).

At time point 202, a patient is presented with symptoms consistent with malignancy. At time point 204 (e.g., −3 weeks), the server 101 acquires a pre-baseline scan 204s, which is a diagnostic scan on which a suspicious lesion was detected. At time point 206, the server 101 acquires (e.g., retrieves, receives) a collection of patient test data on solid tissue, biopsy, and blood biomarkers to confirm or rule-out cancer diagnosis. At time point 208 (e.g., −1 week), the server 101 acquires a Baseline Scan 208s, where the Baseline Scan 208s may be contrast-enhanced CT or PET-CT, and/or may include additional regions (e.g., anatomical structure) with metastatic disease.

At time point 210 (e.g., t=0), the server 101 decides on a treatment plan (e.g., a specific therapy or drug) based on the Pre-Baseline Scan 204s, patient test data, and/or Baseline Scan 208s. The server 101 then starts the patient on the treatment plan.

At time point 212 (e.g., +6 weeks after treatment), the server 101 acquires a 1^{st} follow-up scan 212s, which is an early assessment of the patient's response to the treatment. At time point 214, the server 101 may decide to adjust (e.g., modify) the treatment plan based on radiologic findings in the 1^{st} follow-up scan 212s, or may decide that no adjustment to the treatment plan should be made. In some embodiments, a radiologic finding may include at least one of a tumor growth rate, a tumor volume change, a tumor diameter change, or a tumor shape change, etc. At time point 216, the server 101 acquires a 2^{nd} follow-up scan, which is an assessment of the patient's response to the applied treatment.

### 2.1 Model Training Procedure

The server 101 may train the predictive models according to the following method:

In operation 1, the server 101 creates a collection (e.g., one or more) of training cases, including retrospective longitudinal patient records that include at least one of serial imaging data, medication treatment history, or non-imaging clinical features, etc.

In operation 2, for each training case, the server 101 extracts (e.g., determine, identify) model features and outcome labels (sometimes referred to as, “ground truth”).

In operation 3, the server 101 may extract model features according to the following method:

In operation 3a, the server 101 identifies target lesion(s) on Baseline Scan 208s immediately prior to Treatment Start 210. In operation 3b, the server 101 identifies corresponding target lesions(s) on Pre-Baseline Scan 204s. In operation 3c, the server 101 calculates (e.g., determines, measures) imaging and non-imaging Baseline Features from a Baseline scan 208s. In operation 3d, the server 101 calculates imaging and non-imaging Pre-Baseline Features from Pre-Baseline Scan 204s. In operation 3e, the server 101 calculates a difference or change in imaging and non-imaging features between Pre-Baseline Scan 204s and Baseline Scan 208s. The server 101 may normalize the change in features between Pre-Baseline Scan 204s and Baseline Scan 208s by dividing the change in imaging and non-imaging features by the number of days between the Pre-Baseline Scan 204s and Baseline Scan 208s to produce a normalized change.

In operation 4, the server extracts outcome features according to the following method:

In operation 4a, the server 101 identifies target lesion(s) on Baseline Scan 208s immediately prior to Treatment Start 210.

In operation 4b, the server 101 identifies corresponding target lesions(s) on 1^{st} Follow-up Scan 212s or 2^{nd} Follow-up Scan 216s.

In operation 4c, the server 101 calculates per-lesion response labels for each target lesion. In some embodiments, each label may be one of the following: a categorical variable (e.g., progressive disease, stable disease, partial response, complete response), a scalar variable corresponding to change in diameter, a scalar variable corresponding to absolute change in volume, a scalar variable corresponding to relative (e.g., percent change) in volume, a scalar variable corresponding to growth rate (e.g., linear or exponential change in volume per unit of time).

In operation 4d, the server 101 calculates per-patient response labels using one of the following methods: (a) simple mean (or median) of all per-lesion labels, minimum (or maximum) of all per-lesion labels; (b) categorical variable representing the following states: Uniform response (all target lesions responding to therapy), Uniform progression (all target lesions not responding to therapy and growing), mixed response (some target lesion responding and some progressing); according to known response assessment protocols (e.g. RECIST 1.1, iRECIST, irRECIST, etc.). In some embodiments, other patient-level outcome labels may include at least one of overall survival (e.g., at 6 months, 1 year, 2 years, etc.), change in therapy, treatment discontinuation, or immune-related adverse event, etc.

In some embodiments, the method for calculating features and labels describes the first difference (e.g., velocity) using two time points. An extension of this framework can be constructed where the server 101 uses 3 or more time points to calculate and use second difference (e.g., acceleration) in features and labels.

In some embodiments, the server 101 performs the feature selection method (using known algorithms) to identify a smaller subset of features that most closely associates with the chosen outcome label.

In some embodiments, the server 101 uses an optimization algorithm (e.g., stochastic gradient descent, ADAM, etc.) to train model(s) that, across all training cases, maximize the agreement between outcome labels and model predictions generated from model and its inputs (e.g., features).

### 2.2 Model Inference Procedure

The server 101 may perform a model inference according to the following method:

In operation 1, the server 101 identifies target lesion(s) on Baseline Scan 208s immediately prior to Treatment Start 210. In operation 2, the server 101 identifies corresponding target lesions(s) on Pre-Baseline Scan 204s. In operation 3, the server 101 calculates imaging and non-imaging Baseline Features from Baseline scan 208s. In operation 4, the server 101 calculates imaging and non-imaging Pre-Baseline Features from Pre-Baseline scan 204s.

In operation 5, the server 101 calculate a change in imaging and non-imaging features between Pre-Baseline Scan 204s and Baseline Scan 208s. In some embodiments, a change in features between Pre-Baseline Scan 204s and Baseline Scan 208s are normalized by the number of days between the two scans. For example, the server 101 divides the change in imaging and non-imaging features by the number of days between the Pre-Baseline Scan 204s and Baseline Scan 208s to produce a normalized change.

In operation 6, the server 101 combines Baseline Features, Pre-Baseline Features, and the differences/changes in these features as inputs to lesion-level and patient-level treatment response models to predict treatment response at specific time point after Treatment Start (e.g., +6 weeks, +12 weeks, etc.). In some embodiments, lesion-level models predict treatment response (e.g., growth kinetics) of each individual target lesion. In some embodiments, patient-level model combines predicted growth kinetics of each individual target lesion to combined patient-level response (e.g., in accordance with the RECIST assessment criteria).

In some embodiments, the server 101 may use lesion-level and patient-level predictions to create a treatment plan recommendation.

In some embodiments, the server 101 may collect observed lesion-level and patient-level outcome labels for Online Learning model adaptation.

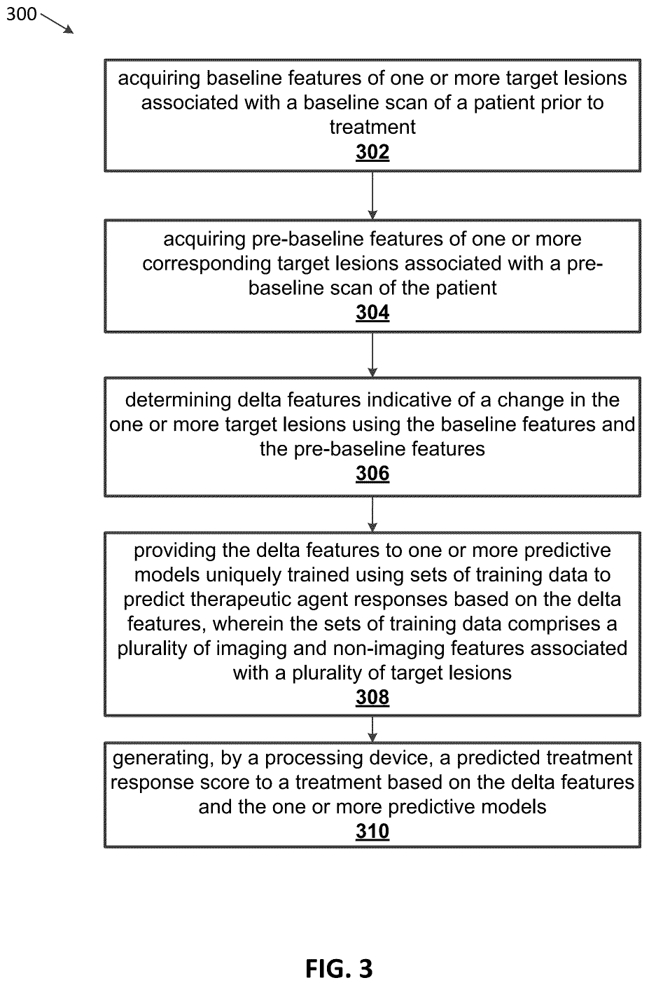

FIG. 3 depicts a flow diagram of a method of predicting therapeutic agent response using deep learning analysis of pre-treatment and intra-treatment serial imaging, according to some embodiments. Each of the methods described herein (including method 300) may be performed by processing logic that may include hardware (e.g., processing device, circuitry, dedicated logic, programmable logic, microcode, hardware of a device, etc.), software (e.g., instructions run or executed on a processing device), or a combination thereof. In some embodiments, the methods may be performed by processing logic of the machine learning architecture 127 of FIG. 1.

As shown in FIG. 3, the method 300 includes the of block 302 acquiring baseline features of one or more target lesions associated with a baseline scan of a patient prior to treatment. In some embodiments, the processing device may acquire the baseline features by retrieving or receiving the baseline features from another computing device. In some embodiments, the processing device may acquire the baseline features by identifying the one or more target lesions on the baseline scan of the patient. A scan (e.g., pre-baseline scans, baseline scans, follow-up scans) may include one or more treatment images. A treatment image may be, but is not limited to, a computed tomography (CT) scan, a positron emission tomography (PET) scan, or a magnetic resonance imaging (MRI) scan. A treatment image from a scan may be a two-dimensional anatomical image, a three-dimensional anatomical image, or a four-dimensional anatomical image. In some embodiments, two or more treatment images of a variety of types (e.g., CT scan, PET scan, MRI scan) may be used.

The method 300 includes the block 304 of acquiring pre-baseline features of one or more corresponding target lesions associated with a pre-baseline scan of the patient. In some embodiments, the processing device may acquire the pre-baseline features by retrieving or receiving the pre-baseline features from another computing device. In some embodiments, the processing device may acquire the pre-baseline features by identifying one or more corresponding target lesions on the pre-baseline scan of the patient.

The method 300 includes the block 306 of determining a set of features indicative of a change in the one or more target lesions using the baseline features and the pre-baseline features. In some embodiments, the processing device may determine the set of features by determining baseline features of the one or more target lesions using a baseline scan of the patient, determining pre-baseline features of the one or more target lesions using a pre-baseline scan of the patient, and comparing (e.g., subtracting) the baseline features and the pre-baseline features to determine a difference. In some embodiments, the processing device may determine a set of features indicative of a change in the one or more target lesions using the baseline features and the pre-baseline features by calculating a difference between the pre-baseline features and the baseline features. The processing device may further normalize the difference to produce a normalized difference by dividing the difference in imaging and non-imaging features by a total number of days between the baseline scan and the pre-baseline scan.

The method 300 includes the block 308 of providing the set of features to one or more deep learning models (sometimes referred to as, “predictive models”) uniquely trained using sets of training data to predict therapeutic agent (e.g., immunotherapy treatment) responses based on the set of features (e.g., changes between serial imaging data from different time points). In some embodiments, the sets of training data may include imaging and/or non-imaging features associated with target lesions of a plurality of patients. In some embodiments, the sets of training data may include information indicating one or more changes in lesion volume and/or lesion diameter, and/or other patient-level endpoints including, for example, progression-free survival (PFS), overall survival (OS), clinical benefit, and objective response per RECIST protocol. Examples of a deep learning model include, but are not limited to, artificial neural network, convolutional neural networks, random forest model, support vector machine, and logistic regression model. In another embodiment, a single deep learning model may be used. In some embodiments, the one or more predictive models are trained using training data that includes a plurality of imaging and non-imaging features associated with a plurality of target lesions of a plurality of patients. In some embodiments, the one or more predictive models are further trained to predict the therapeutic agent responses based on a change in lesion volume.

In some embodiments, a computing system may train a predictive model to predict a therapeutic agent response using one or more sets of training data, as described herein. In some embodiments, the computing system may train, using one or more sets of training data, a predictive model to predict a therapeutic agent response that is indicative of pseudo-progression based on at least one or more of a change in volume of a lesion of a patient or a change in diameter of the lesion of the patient. In some embodiments, the computing system improves a prediction accuracy of a predictive model by training the predictive model with a plurality of pre-treatment scans associated with a plurality of patients.

The deep learning models may utilize a variety of suitable training methods as discussed herein. For example, in one embodiment, the deep learning models use a population of training subjects and a plurality of images associated with each of a plurality of training subjects as training data. In another embodiment, the deep learning models use calculated subject-specific models as training data. In yet another embodiment, the deep learning models use a combination of the two methods described above. In another embodiment, the models are trained on different data, using different techniques, have different objectives, etc., the results of which may be aggregated in a variety of ways.

In one embodiment, the treatment is a PD-1-based treatment. In another embodiment, the treatment is a PD-L1-based treatment. In yet another embodiment, the treatment is a CTLA-4-based treatment, or any other suitable treatment type (e.g., chemotherapy, pharmaceutical-based therapy, radiotherapy, etc.).

The method 300 includes the block 310 of generating, by a processing device, a predicted treatment response score (e.g., on a scale representing least likely to have a positive of negative effect to most likely to have a positive or negative effect) to a treatment based on the set of features and the one or more deep learning models. In one embodiment, processing logic generates the predicted treatment response score based on the single pre-treatment image and the two or more deep learning models. For example, in one embodiment, results from the different models may be combined (e.g., averaged, or combined in any other way) to generate a single response score.

In one embodiment, the predicted treatment response score includes a prediction of patient progression on a predefined pharmaceutical product. In another embodiment, the predicted treatment response score indicates a prediction of one or more immune-related adverse events associated with the immunotherapy treatment. In one embodiment, the predicted treatment response score may include a predicted likelihood (e.g., a confidence level) of a specific type of response and/or adverse event occurring. In another embodiment, the response score may also include an indication of pseudo-progression, which is characterized by short-term and temporary increase in tumor volume due to natural swelling and/or inflammation (e.g., in response to treatment), rather than progression of disease. In another embodiment, the response score may indicate the likelihood of hyper-progression, which is a serious condition in which progression of disease is accelerated by administration of therapy. In another embodiment, the response score may include an indication of pseudo-progression, which is characterized by short-term and temporary increase in tumor volume due to at least one of natural swelling or natural inflammation (e.g., in response to treatment), rather than progression of a disease. In another embodiment, the response score may be formulated to indicate progression-free or overall patient survival in units of months or years. In another embodiment, generating the predicted treatment response score is further based on pre-treatment information indicative of at least one of: a change in blood lab values, a change in urine lab values, or a change in imaging features.

In some embodiments, the processing device may acquire post-treatment features of the one or more target lesions associated with a post-treatment scan of the patient after treating the patient according to the treatment plan. In some embodiments, the processing device may determine a second set of features indicative of a second change in the one or more target lesions using the post-treatment features and at least one of the baseline features or the pre-treatment features. In some embodiments, the processing device may provide the second set of features to the one or more predictive models. In some embodiments, the processing device may generate a second predicted treatment response score to a second treatment plan for the patient based on the second set of features and the one or more predictive models after treating the patient according to the treatment plan.

The method 300 may include the block 316 (not shown in FIG. 3) of providing, based on the predicted treatment response, a recommended treatment plan. For example, based on the predicted treatment response, a recommended treatment plan may include an indication of whether a specific pharmaceutical product should be used, a dosage of such product, a timing associated with administering such a product, etc. In one embodiment, the per-lesion immunotherapy and/or chemotherapy response predictions are used to generate a lesion-specific therapy plan to enhance the therapeutic effect in high-risk lesions by combining ongoing systemic therapy with localized therapy. Localized therapy may be any of the following: stereotactic ablative radiation therapy (SBRT), intensity modulated radiation therapy (IMRT), conformal radiation therapy (CRT), radiosurgery, surgical resection, thermal ablation, cryoablation, or high intensity focused ultrasound (HIFU) therapy. In another embodiment, the recommended treatment plan may be to discontinue one or all therapeutic methods to maximize patient's quality of life.

In one embodiment, processing logic may perform a variety of follow-up operations to increase the accuracy of at least one of the prediction or recommendation. For example, in one embodiment, processing logic may receive an intra-treatment follow-up image, provide the intra-treatment follow-up image to the machine learning model, and generate an updated predicted treatment response score. Processing logic may then provide, based on the updated predicted treatment response score, an updated recommended treatment plan. In one embodiment, the pre-treatment image and intra-treatment follow-up image each comprise a plurality of imaging-based biomarkers.

In a variety of embodiments, processing logic may perform any number of suitable pre- and post-processing operations that may increase the accuracy, efficiency, and/or compatibility of the machine learning model in the context at hand. For example, with respect to preprocessing, traditional radiomics methods may be susceptible to variations in scanner hardware and imaging protocols. The data preprocessing and data augmentation systems described herein are designed to optimize model generalizability and to minimize model susceptibility to imaging hardware and protocol variations.

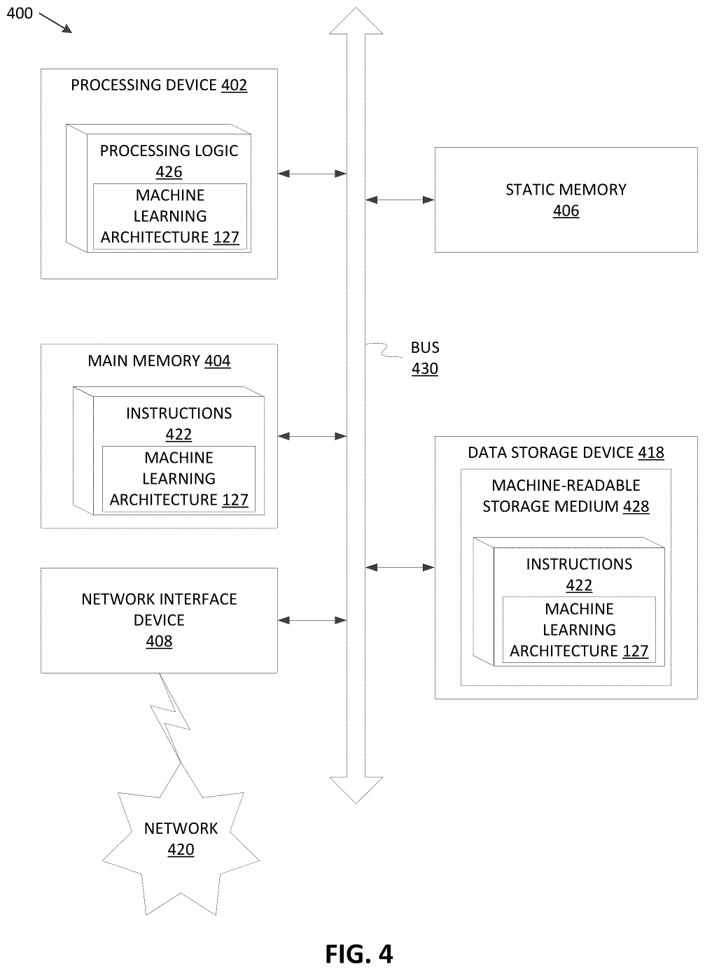

FIG. 4 illustrates a diagrammatic representation of a machine in the example form of a computer system 400 within which a set of instructions 422, for causing the machine to perform any one or more of the methodologies discussed herein, may be executed. In alternative embodiments, the machine may be connected (e.g., networked) to other machines in a local area network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, a switch or bridge, a hub, an access point, a network access control device, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. In one embodiment, computer system 400 may be representative of a server computer system, such as system 100.

The exemplary computer system 400 includes a processing device 402, a main memory 404 (e.g., read-only memory (ROM), flash memory, dynamic random-access memory (DRAM), a static memory 406 (e.g., flash memory, static random-access memory (SRAM), etc.), and a data storage device 418, which communicate with each other via a bus 430. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses.

Processing device 402 represents one or more general-purpose processing devices such as a microprocessor, central processing unit, or the like. More particularly, the processing device may be complex instruction set computing (CISC) microprocessor, reduced instruction set computer (RISC) microprocessor, very long instruction word (VLIW) microprocessor, or processor implementing other instruction sets, or processors implementing a combination of instruction sets. Processing device 402 may also be one or more special-purpose processing devices such as an application specific integrated circuit (ASIC), a field programmable gate array (FPGA), a digital signal processor (DSP), network processor, or the like. The processing device 402 is configured to execute processing logic 426, which may be one example of system 100 shown in FIG. 1, for performing the operations and steps discussed herein.

The data storage device 418 may include a machine-readable storage medium 428, on which is stored one or more set of instructions 422 (e.g., software) embodying any one or more of the methodologies of functions described herein, including instructions to cause the processing device 402 to execute system 100. The instructions 422 may also reside, completely or at least partially, within the main memory 404 or within the processing device 402 during execution thereof by the computer system 400; the main memory 404 and the processing device 402 also constituting machine-readable storage media. The instructions 422 may further be transmitted or received over a network 420 via the network interface device 408.

The machine-readable storage medium 428 may also be used to store instructions to perform the methods and operations described herein. While the machine-readable storage medium 428 is shown in an exemplary embodiment to be a single medium, the term “machine-readable storage medium” should be taken to include a single medium or multiple media (e.g., a centralized or distributed database, or associated caches and servers) that store the one or more sets of instructions. A machine-readable medium includes any mechanism for storing information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; or another type of medium suitable for storing electronic instructions.

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format to avoid unnecessarily obscuring the present disclosure. Thus, the specific details set forth are merely exemplary. Particular embodiments may vary from these exemplary details and still be contemplated to be within the scope of the present disclosure.

Additionally, some embodiments may be practiced in distributed computing environments where the machine-readable medium is stored on and or executed by more than one computer system. In addition, the information transferred between computer systems may either be pulled or pushed across the communication medium connecting the computer systems.

Embodiments of the claimed subject matter include, but are not limited to, various operations described herein. These operations may be performed by hardware components, software, firmware, or a combination thereof.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent or alternating manner.

The above description of illustrated implementations of the present disclosure, including what is described in the Abstract, is not intended to be exhaustive or to limit the present disclosure to the precise forms disclosed. While specific implementations of, and examples for, the present disclosure are described herein for illustrative purposes, various equivalent modifications are possible within the scope of the present disclosure, as those skilled in the relevant art will recognize. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “of” is intended to mean an inclusive “or” rather than an exclusive “of”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

It will be appreciated that variants of the above-disclosed and other features and functions, or alternatives thereof, may be combined into may other different systems or applications. Various presently unforeseen or unanticipated alternatives, modifications, variations, or improvements therein may be subsequently made by those skilled in the art which are also intended to be encompassed by the following claims. The claims may encompass embodiments in hardware, software, or a combination thereof. In the foregoing specification, the disclosure has been described with reference to specific exemplary implementations thereof. It will, however, be evident that various modifications and changes may be made thereto without departing from the broader spirit and scope of the disclosure as set forth in the appended claims. The specification and drawings are, accordingly, to be regarded in an illustrative sense rather than a restrictive sense.

What is claimed, is:

1. A method, comprising:
acquiring pre-treatment features of one or more target lesions associated with a pre-treatment scan of a target subject prior to treating the target subject according to a treatment plan;
determining a set of features indicative of a change in the one or more target lesions using the pre-treatment features;
providing the set of features to one or more predictive models trained to predict therapeutic agent responses based on features of target lesions; and
generating, by a processing device, a predicted treatment response score for the treatment plan based on the set of features and the one or more predictive models prior to treating the target subject according to the treatment plan.

2. The method of claim 1, wherein the one or more predictive models are trained using training data comprising a plurality of imaging and non-imaging features associated with a plurality of target lesions of a plurality of target subjects.

3. The method of claim 1, wherein generating the predicted treatment response score is further based on pre-treatment information indicative of at least one of:
a change in blood lab values,
a change in urine lab values, or
a change in imaging features.

4. The method of claim 1, wherein the one or more predictive models are further trained to predict the therapeutic agent responses based on a change in lesion volume.

5. The method of claim 1, further comprising:
improving a prediction accuracy of the one or more predictive models by training the one or more predictive models with a plurality of pre-treatment scans associated with a plurality of target subjects.

6. The method of claim 5, wherein acquiring the pre-treatment features of the one or more target lesions associated with the pre-treatment scan of the target subject further comprises:
acquiring baseline features of one or more target lesions associated with a baseline scan of the target subject prior to administering the treatment plan to the target subject; and
acquiring pre-baseline features of one or more corresponding target lesions associated with a pre-baseline scan of the target subject prior to administering the treatment plan to the target subject.

7. The method of claim 6, wherein determining the set of features indicative of the change in the one or more target lesions using the pre-treatment features further comprises:
calculating a difference between the pre-baseline features and the baseline features.

8. The method of claim 7, further comprising:
normalizing the difference to produce a normalized difference by dividing the difference in imaging and non-imaging features by a total number of days between the baseline scan and the pre-baseline scan.

9. The method of claim 1, further comprising:
acquiring post-treatment features of the one or more target lesions associated with a post-treatment scan of the target subject after treating the target subject according to the treatment plan;
determining a second set of features indicative of a second change in the one or more target lesions using the post-treatment features and at least one of the baseline features or the pre-treatment features;
providing the second set of features to the one or more predictive models; and
generating a second predicted treatment response score to a second treatment plan for the target subject based on the second set of features and the one or more predictive models after treating the target subject according to the treatment plan.

10. The method of claim 1, wherein the predicted treatment response score comprises:
an indication of pseudo-progression associated with the one or more target lesions,
an indication of hyper-progression associated with the one or more target lesions, or
an indication of overall target subject survival.

11. A treatment analysis system comprising:
a memory to store a pre-treatment scan of a target subject; and
a processing device, operatively coupled to the memory, the processing device to:
acquire pre-treatment features of one or more target lesions associated with the pre-treatment scan of the target subject prior to treating the target subject according to a treatment plan;
determine a set of features indicative of a change in the one or more target lesions using the pre-treatment features;
provide the set of features to one or more predictive models trained to predict therapeutic agent responses based on features of target lesions; and
generate a predicted treatment response score for the treatment plan based on the set of features and the one or more predictive models prior to treating the target subject according to the treatment plan.

12. The treatment analysis system of claim 11, wherein the one or more predictive models are trained using training data comprising a plurality of imaging and non-imaging features associated with a plurality of target lesions of a plurality of target subjects.

13. The treatment analysis system of claim 11, wherein to generate the predicted treatment response score is further based on pre-treatment information indicative of at least one of:
a change in blood lab values,
a change in urine lab values, or
a change in imaging features.

14. The treatment analysis system of claim 11, wherein the one or more predictive models are further trained to predict the therapeutic agent responses based on a change in lesion volume.

15. The treatment analysis system of claim 11, wherein the processing device is further to:
improve a prediction accuracy of the one or more predictive models by training the one or more predictive models with a plurality of pre-treatment scans associated with a plurality of target subjects.

16. The treatment analysis system of claim 15, wherein to acquire the pre-treatment features of the one or more target lesions associated with the pre-treatment scan of the target subject, the processing device is further to:
acquire baseline features of one or more target lesions associated with a baseline scan of the target subject prior to administering the treatment plan to the target subject; and
acquire pre-baseline features of one or more corresponding target lesions associated with a pre-baseline scan of the target subject prior to administering the treatment plan to the target subject.

17. The treatment analysis system of claim 16, wherein to determine the set of features indicative of the change in the one or more target lesions using the pre-treatment features, the processing device is further to:
calculate a difference between the pre-baseline features and the baseline features; and
normalize the difference to produce a normalized difference by dividing the difference in imaging and non-imaging features by a total number of days between the baseline scan and the pre-baseline scan.

18. The treatment analysis system of claim 11, wherein the processing device is further to:
acquire post-treatment features of the one or more target lesions associated with a post-treatment scan of the target subject after treating the target subject according to the treatment plan;
determine a second set of features indicative of a second change in the one or more target lesions using the post-treatment features and at least one of the baseline features or the pre-treatment features;
provide the second set of features to the one or more predictive models; and
generate a second predicted treatment response score to a second treatment plan for the target subject based on the second set of features and the one or more predictive models after treating the target subject according to the treatment plan.

19. The treatment analysis system of claim 11, wherein the predicted treatment response score comprises:
an indication of pseudo-progression associated with the one or more target lesions,
an indication of hyper-progression associated with the one or more target lesions, or
an indication of overall target subject survival.

20. A non-transitory computer-readable storage medium comprising instructions, which when executed by a processing device, cause the processing device to:
acquire pre-treatment features of one or more target lesions associated with a pre-treatment scan of a target subject prior to treating the target subject according to a treatment plan;
determine a set of features indicative of a change in the one or more target lesions using the pre-treatment features;
provide the set of features to one or more predictive models trained to predict therapeutic agent responses based on features of target lesions; and
generate, by the processing device, a predicted treatment response score for the treatment plan based on the set of features and the one or more predictive models prior to treating the target subject according to the treatment plan.
